(19) United States
(12) Patent Application Publication
(10) Pub. No.: US 2026/0339436 A1
(43) Pub. Date: Oct. 29, 2026

(54) Eigen-Cluster Neuro-Interface for Qualia Mapping

(71) Applicant: Jonathan Washburn, Austin, TX (US)

(72) Inventor: Jonathan Washburn, Austin, TX (US)

(21) Appl. No.: 19/652,830

(22) Filed: Apr. 20, 2026

Related U.S. Application Data
(60) Provisional application No. 63/794,448, filed on Apr. 25, 2025.

(51) Int. Cl. G06F 3/01; A61B 5/381; A61B 5/383

(52) U.S. Cl. CPC G06F 3/015; A61B 5/381; A61B 5/383

(57) ABSTRACT

A neural interface for subjective qualia is disclosed. Cortical neural data representing a neural field are obtained from a subject and decomposed into coefficients in a fixed Recognition-Physics-derived cortical eigen-basis. A qualia state is identified from the coefficients according to a deterministic mapping without subject-specific training data for qualia classification. In certain embodiments, a target qualia state is represented by a target coefficient vector, and a stimulation signal is generated for writing the target qualia state in the same cortical eigen-basis. In preferred embodiments, the cortical eigen-basis comprises eight modes associated with an 8-tick Recognition structure. In certain embodiments, subject-specific variation is limited to anatomical embedding and stimulation-coupling calibration. The disclosed systems and methods provide a training-free, basis-consistent architecture for qualia readout and qualia-directed cortical stimulation.

### CROSS-REFERENCE TO RELATED APPLICATIONS

This application claims priority to U.S. Provisional Patent Application No. 63/794,448, filed on Apr. 25, 2025, the entire disclosure of which is hereby incorporated by reference herein in its entirety.

### BACKGROUND

The present disclosure relates generally to neural signal processing, brain-computer interfaces, cortical electrophysiology, cortical stimulation, and computational analysis of subjective qualia.

Brain-computer interfaces are commonly used to acquire, process, classify, and in some cases stimulate neural activity associated with sensory, motor, cognitive, or affective states. Existing systems frequently analyze neural activity using frequency-band decompositions, spatial voxel representations, principal-component techniques, independent-component techniques, classifier-derived latent spaces, or other statistical representations derived from recorded neural data.

While such approaches can be useful for signal compression, state classification, motor decoding, workload estimation, seizure monitoring, and related applications, such approaches do not inherently provide a principled subject-independent basis for mapping cortical activity to subjective qualia. Instead, many conventional systems depend on learned relationships between measured neural signals and reported subjective states.

In many existing neural decoding approaches, mappings between neural measurements and perceived sensory, affective, or experiential states are obtained using labeled training data, subject-specific decoder fitting, repeated recalibration, session-specific adjustment, or statistical inference. As a result, such systems may vary across subjects, may vary across sessions, and may drift over time.

A further difficulty in existing systems is that read operations and write operations, when both are present, may be performed in different representational spaces. For example, a decoder may interpret recorded neural activity in one learned or statistical space, while a stimulation system may apply stimulation according to a different anatomical, hardware-defined, or empirically tuned space. Such mismatch can complicate interpretation, control, reproducibility, and closed-loop operation.

Existing approaches also may require substantial subject-specific data collection, repeated model adaptation, or empirically derived mappings to associate measured cortical activity with reported subjective states. In settings where direct qualia interpretation or qualia-directed stimulation is desired, such dependence on subject-specific training and repeated adaptation can create operational complexity and reduce portability across sessions and subjects.

Accordingly, there remains a need for improved neural interfacing systems and methods that provide a principled basis for relating cortical neural activity to subjective qualia, reduce or eliminate dependence on subject-specific qualia-model training, and support more coherent read and write operations for qualia-related neural states.

In some embodiments, the disclosed technology improves computer-controlled neural interfacing not by merely presenting an interpretation of neural data, but by imposing a fixed machine-executable basis architecture that is used for acquisition-side projection, coefficient generation, deterministic state identification, target-state definition, stimulation synthesis, normalization, and closed-loop update. In representative implementations, this architecture reduces dependence on repeated subject-specific decoder retraining, reduces mismatch between read-space and write-space representations, and enables basis-consistent machine control of stimulation output using a common coefficient-space representation. These technical effects arise from the disclosed fixed-basis projection and control architecture rather than from presentation of information alone.

### SUMMARY

The present disclosure provides neural interfacing systems and methods for reading and, in certain embodiments, writing subjective qualia using a fixed Recognition-Physics-derived cortical eigen-basis. In the disclosed architecture, cortical neural activity is acquired from a subject, represented in a predefined cortical eigen-basis, and converted into a coefficient vector from which a qualia state is determined by deterministic mapping without subject-specific training data for qualia classification.

In one aspect, a neural acquisition subsystem obtains cortical neural data representing a neural field u(x, t). A projection subsystem represents the neural field in a fixed Recognition-Physics-derived cortical eigen-basis comprising projected cortical eigen-modes psi_k. A readout subsystem determines coefficients w_k(t) corresponding to the neural field in that basis and determines a qualia state from the resulting coefficient vector w(t) by deterministic mapping. In preferred implementations, the same basis is also used for write operations.

In certain embodiments, a target qualia state is represented by a target coefficient vector w_target. A stimulation subsystem generates cortical stimulation in the same eigen-basis used for readout, thereby enabling basis-consistent read/write coupling. In preferred embodiments, subject-specific adaptation is limited to anatomical embedding and stimulation-coupling calibration rather than subject-specific qualia-model training.

In preferred implementations, the neural field may be represented as follows:

u(x,t)=sum_k w_k(t) psi_k(x)

w_k(t)=integral over M_cortex of u(x,t) psi_k(x) dx

sum_k|w_k(t)|\hat{ }2=1

I(x,t)=sum_k s_k(t) psi_k\hat{ }cortex(x)

s_k(t)=G_k*w_target_k(t)

In preferred implementations, the disclosed basis comprises eight modes tied to a stated 8-tick Recognition structure. The disclosed interface may therefore provide a fixed, non-statistical representational basis for qualia-related readout and stimulation, rather than a learned subject-specific latent space. In narrower embodiments, the coefficient vector is normalized according to a unitarity or unit-sphere convention for stable control and interpretation.

In certain embodiments, anatomical embedding data are used to map universal eigen-modes onto a subject-specific cortical geometry. In certain embodiments, a one-time stimulation-coupling calibration is performed to determine a coupling matrix G for later use in stimulation synthesis. In such embodiments, the disclosed interface remains training-free with respect to qualia classification even though anatomical and coupling measurements may be obtained for a given subject.

In some embodiments, the disclosed systems operate in read-only mode. In some embodiments, the disclosed systems operate in write-only mode. In some embodiments, the disclosed systems operate in a combined read/write mode. In some embodiments, the disclosed systems operate in a closed-loop mode in which a measured coefficient vector is compared to a target coefficient vector and stimulation is updated based on the difference between the two.

In preferred embodiments, the disclosed architecture provides one or more of the following advantages: training-free qualia classification; deterministic mapping between coefficient vectors and qualia states; same-basis read and write operations; universal eigen-modes with subject-specific adaptation limited to anatomy and coupling calibration; sensor-agnostic implementation across multiple sensing modalities; and optional closed-loop normalization and safety monitoring.

Additional aspects, embodiments, implementations, and advantages will be apparent from the following description, the drawings, and the claims.

Although the disclosed architecture may be implemented across multiple sensing modalities, stimulation modalities, deployment modes, and downstream applications, the present disclosure does not require every embodiment to support every modality, every write pathway, every downstream application, every channel count, every validation target, or every subject population. In some embodiments, the disclosed interface is implemented only in a read-only mode. In some embodiments, the disclosed interface is implemented only in a write-capable mode after coupling calibration. In some embodiments, narrower implementations are limited to selected qualia categories, selected sensing arrangements, selected stimulation arrangements, or selected control modes while preserving the disclosed common inventive concept of fixed-basis, training-free, basis-consistent qualia interfacing.

### BRIEF DESCRIPTION OF THE DRAWINGS

FIG. 1 is an overall block diagram of an Eigen-Cluster Neuro-Interface system showing neural acquisition, fixed-basis projection, deterministic qualia readout, same-basis write-path stimulation synthesis, calibration storage, control logic, safety monitoring, and output products.

FIG. 2 is a flowchart of a read-side workflow from cortical signal acquisition through preprocessing, basis projection, coefficient generation, and deterministic qualia classification.

FIG. 3 is a schematic diagram of an eigen-cluster extraction pipeline showing sensor-space neural data, covariance generation, joint diagonalization with a precomputed Recognition-Physics basis, and coefficient vector output.

FIG. 4 is a schematic diagram of anatomical embedding showing mapping of universal eigen-modes onto a subject-specific cortical geometry.

FIG. 5 is a schematic diagram of deterministic qualia readout showing coefficient ranking, dominant-mode identification, fixed qualia mapping, and optional sub-classification.

FIG. 6 is a schematic diagram of a write-side pathway showing target qualia definition, target coefficient vector formation, coupling calibration, basis-pattern synthesis, and cortical stimulation output.

FIG. 7 is a schematic diagram of a closed-loop control and safety process showing read, compare, normalize, update, stimulate, and safety-threshold operations.

FIG. 8 is a schematic diagram of a mathematical framework showing a projected cortical operator, eigen-mode relationships, projector structure, and residual decomposition outside a disclosed basis.

FIG. 9 is a schematic diagram showing representative sensing and stimulation embodiments, including invasive and non-invasive sensing arrangements and multiple stimulation modalities.

FIG. 10 is a schematic diagram showing representative downstream embodiments, including pain modulation, neuro-prosthetic feedback, immersive media, and monitoring or diagnostic applications.

FIG. 11 is a schematic diagram showing validation and support architecture, including formal verification, validation protocols, prophetic material, and future continuation targets.

### DETAILED DESCRIPTION

The following detailed description sets forth illustrative embodiments of the disclosed systems and methods for reading and, in certain embodiments, writing subjective qualia using a fixed Recognition-Physics-derived cortical eigen-basis. The detailed description is provided to enable a person of ordinary skill in the relevant art to make and use the disclosed subject matter and is not intended to limit the scope of the claims.

As used herein, the term “neural field” refers to cortical neural activity represented as a spatially and temporally varying signal u(x,t), whether treated as a continuous field over cortical tissue or as a discrete signal sampled over sensor locations, channels, electrodes, voxels, or reconstructed cortical positions.

As used herein, the term “Recognition-Physics operator” refers to an operator H_RP used to define the fixed basis employed by the disclosed interface. As used herein, the term “projected cortical operator” refers to an operator H_cortex obtained by projection or embedding of the Recognition-Physics operator onto a subject-specific cortical geometry.

As used herein, the term “projected cortical eigen-mode” refers to a basis function psi_k\hat{ }cortex defined on a cortex and corresponding to a projected Recognition-Physics mode. As used herein, the term “coefficient vector” refers to a vector w(t) comprising coefficients w_k(t) representing the neural field in the disclosed cortical eigen-basis.

As used herein, the term “qualia state” refers to a basis-defined subjective state represented by one or more coefficients in the disclosed cortical eigen-basis. As used herein, the term “target qualia state” refers to a desired subjective state represented by a target coefficient vector w_target.

As used herein, the term “training-free” means that no subject-specific training data is used to learn a qualia classifier, decoder, or mapping between neural data and qualia categories. Anatomical embedding and stimulation-coupling calibration, as described herein, are not qualia-model training.

As used herein, the term “anatomical embedding” refers to a mapping iota between an abstract Recognition-Physics mode structure and a subject-specific cortical geometry. As used herein, the term “coupling matrix” refers to a calibration-derived mapping G between target coefficient values and stimulation parameters used to generate basis-consistent stimulation.

As used herein, the term “unitarity” or “normalized coefficient convention” refers to a convention in which a coefficient vector is normalized, for example by enforcing sum_k|w_k(t)|\hat{ }2=1. As used herein, the term “qualia track” refers to a stored, replayable, analyzable, or transmissible record of coefficient vectors, target vectors, stimulation patterns, or related basis-defined qualia representations.

In one embodiment, system 100 comprises a neural acquisition subsystem 104, a preprocessing subsystem 108, a basis datastore 112, a projection subsystem 114, a deterministic readout subsystem 118, a stimulation subsystem 124, a calibration subsystem 122, a controller 126, a safety monitor 128, and an output datastore 130. In some embodiments, system 100 further includes a user interface 132 and compute hardware 134.

Neural acquisition subsystem 104 is configured to obtain cortical neural data from a subject cortex 102. In some embodiments, neural acquisition subsystem 104 comprises an invasive sensing arrangement such as an electrocorticography array. In some embodiments, neural acquisition subsystem 104 comprises a non-invasive sensing arrangement such as electroencephalography, magnetoencephalography, functional near-infrared spectroscopy, or another neural sensing arrangement capable of acquiring cortical neural data.

Preprocessing subsystem 108 is configured to receive neural data from neural acquisition subsystem 104 and perform one or more preprocessing operations, including synchronization, filtering, denoising, segmentation, registration, normalization, artifact suppression, channel alignment, or other preparation steps suitable for subsequent basis projection.

Basis datastore 112 is configured to store a predefined Recognition-Physics-derived cortical eigen-basis or data sufficient to generate such basis. In preferred embodiments, the stored basis comprises an eight-mode implementation tied to a stated 8-tick Recognition structure. In some embodiments, basis datastore 112 stores mode definitions, eigenvalue ordering data, projector data, anatomical embedding information, or combinations thereof.

Projection subsystem 114 is configured to represent measured cortical neural activity in the fixed cortical eigen-basis and to generate a coefficient vector w(t). In certain embodiments, projection subsystem 114 computes coefficients directly from an inner product or discrete summation. In certain embodiments, projection subsystem 114 computes coefficients using covariance generation and joint diagonalization relative to the predefined basis.

Deterministic readout subsystem 118 is configured to determine a qualia state from coefficient vector w(t) according to a deterministic mapping. In preferred embodiments, deterministic readout subsystem 118 identifies a dominant mode and maps the dominant mode to a qualia category according to a fixed basis-defined mapping. In certain embodiments, deterministic readout subsystem 118 further performs sub-classification using additional coefficient information.

Stimulation subsystem 124 is configured, in write-capable embodiments, to generate cortical stimulation corresponding to a target coefficient vector w_target in the same basis used by projection subsystem 114 and deterministic readout subsystem 118. In preferred embodiments, stimulation subsystem 124 synthesizes a stimulation pattern as a weighted combination of projected cortical eigen-modes.

Calibration subsystem 122 is configured to store and, in some embodiments, generate subject-specific calibration data. In preferred embodiments, such calibration data include anatomical embedding information and stimulation-coupling information. In preferred embodiments, subject-specific adaptation is limited to such anatomical and coupling calibration rather than subject-specific qualia-model training.

Controller 126 is configured, in certain embodiments, to coordinate read, compare, normalize, update, and stimulate operations in a combined read/write system. Safety monitor 128 is configured to evaluate one or more safety conditions, including normalization deviation, stimulation amplitude limits, or other threshold conditions, and to trigger one or more protective operations, including limiting stimulation, pausing stimulation, or shutting down stimulation output.

Output datastore 130 is configured to store one or more outputs of system 100, including coefficient vectors, qualia classifications, stimulation patterns, calibration records, validation records, safety logs, and qualia tracks. In some embodiments, user interface 132 is configured to display or receive operator input relating to target qualia states, validation workflows, stimulation sessions, monitoring functions, or stored output products.

System 100 may be operated in a read-only mode, a write-only mode, a combined read/write mode, or a closed-loop mode. In a read-only mode, the system acquires cortical neural activity, projects the activity into the fixed basis, and determines a qualia state. In a write-only mode, the system receives a target qualia state or target coefficient vector and generates stimulation in the disclosed basis. In a combined read/write mode, the system performs both operations. In a closed-loop mode, the system updates stimulation based on a measured difference between a current coefficient vector and a target coefficient vector.

The disclosed architecture thereby provides a basis-consistent neural interface in which cortical activity is represented, interpreted, and optionally stimulated in a common Recognition-Physics-derived eigen-basis without requiring subject-specific training data for qualia classification.

In preferred embodiments, the disclosed interface operates in a fixed Recognition-Physics-derived cortical eigen-basis. In such embodiments, a Recognition-Physics operator H_RP defines an abstract mode structure, and a projected cortical operator H_cortex is defined by projection or embedding of H_RP onto a subject-specific cortical geometry.

In one preferred implementation, the projected cortical operator is represented in plain text as follows:

H_cortex=iota* H_RP iota

where iota denotes an anatomical embedding between an abstract Recognition-Physics mode structure and a subject-specific cortical geometry, and iota* denotes a corresponding pullback, projection, or equivalent mapped operator representation.

In preferred embodiments, the projected cortical eigen-modes satisfy the following eigen-equation:

H_cortex psi_k=lambda_k psi_k

where psi_k denotes a projected cortical eigen-mode and lambda_k denotes a corresponding eigenvalue.

In the disclosed implementation, the basis comprises eight modes associated with a stated 8-tick Recognition structure. In preferred embodiments, the eigenvalue ordering is represented in plain text as follows:

lambda_k=J(phi\hat{ }k)=0.5(phi\hat{ }k+phi\hat{ }(−k))−1

where J denotes a Recognition cost function and phi denotes a stated Recognition scaling parameter. The foregoing expression is provided as a preferred implementation of mode ordering and basis structure in the disclosed system.

In preferred embodiments, the neural field u(x,t) is represented in the disclosed basis according to the following expression:

u(x,t)=sum_k w_k(t) psi_k(x)

where w_k(t) are coefficients representing the contribution of each projected cortical eigen-mode psi_k to the measured neural field.

In preferred embodiments, the coefficient corresponding to a given mode is obtained according to the following expression:

w_k(t)=integral over M_cortex of u(x,t) psi_k(x) dA(x)

where M_cortex denotes the cortical manifold and dA(x) denotes an area element or equivalent spatial weighting term.

In discrete implementations, the foregoing integral may be approximated over sensor locations, electrodes, reconstructed cortical positions, or other sampled positions according to the following expression:

w_k(t) approx equals sum_i u(x_i,t) psi_k(x_i) Delta A_i

where x_i denotes a sampled position and Delta A_i denotes an associated area, weight, or discretization factor.

In preferred embodiments, the interface operates on a projected component of the neural field lying in the disclosed basis and treats activity outside that basis as a residual or orthogonal complement. In one preferred representation, a projector onto the disclosed subspace is given by:

Pi_8(x,x′)=sum_k psi_k(x) psi_k(x′)

and the measured field may be separated into a projected component and a residual component.

In preferred embodiments, the disclosed interface uses a rank-8 projected subspace associated with the eight-mode implementation described herein. Activity represented by the projected component may be used for deterministic readout, stimulation synthesis, normalization, control, and validation, while activity outside the projected subspace may be treated as residual activity, noise, artifact, out-of-basis activity, or a quality-control quantity.

In certain embodiments, the residual component is monitored as part of a quality metric. For example, an unusually large residual component, an unusually large deviation from a normalized coefficient convention, or an unusually poor reconstruction of the measured field from the projected coefficients may indicate measurement artifact, poor registration, drift, pathological cortical state, inadequate calibration, or another condition warranting rejection, flagging, recalibration, or safety intervention.

In preferred embodiments, anatomical embedding data are used to map universal eigen-modes onto a subject-specific cortical geometry. In one preferred implementation, the mapping is represented in plain text as follows:

psi_k\hat{ }cortex(x)=psi_k(iota\hat{ }(−1)(x))

where psi_k denotes an abstract Recognition-Physics mode and psi_k\hat{ }cortex denotes the corresponding projected cortical eigen-mode.

In preferred embodiments, anatomical embedding information is derived from anatomical imaging data, including but not limited to magnetic resonance imaging data. In other embodiments, anatomical embedding information may be derived from source localization data, cortical surface reconstruction data, atlas registration data, or equivalent anatomical or geometric data sufficient to map the disclosed basis onto cortical tissue.

In preferred embodiments, the eigen-modes themselves are universal, and subject-specific variation is limited to anatomical embedding and stimulation-coupling calibration. Accordingly, the disclosed interface remains training-free with respect to qualia classification even though anatomical data may be obtained for a given subject.

In certain embodiments, projection subsystem 114 generates the coefficient vector from preprocessed neural data using an inner-product computation, a matrix projection, a spectral decomposition, or another basis-projection process. In preferred embodiments, projection subsystem 114 performs a covariance-based extraction process.

In one preferred implementation, a spatiotemporal covariance matrix is computed according to the following expression:

C_ij(tau)=average over t of u(x_i,t)*u(x_j, t+tau)

where x_i and x_j denote sensor locations, reconstructed cortical positions, or equivalent sampled positions, and tau denotes a temporal lag.

In preferred embodiments, the coefficient extraction pipeline uses joint diagonalization of the covariance matrix with respect to the disclosed Recognition-Physics basis. In one preferred implementation, a transform matrix W is obtained according to the following expression:

W=argmin_W∥W\hat{ }T C W−Lambda∥_F\hat{ }2

subject to:

W\hat{ }T H_RP W=I

where Lambda denotes a diagonal or substantially diagonal target representation and I denotes an identity or orthonormality constraint.

In preferred embodiments, the resulting transform is used to generate the coefficient vector according to the following expression:

w(t)=W\hat{ }T u(t)

where u(t) denotes a vectorized neural signal representation at time t.

In certain embodiments, the extraction pipeline includes one or more preprocessing operations before coefficient generation, including synchronization, filtering, denoising, artifact suppression, channel weighting, spatial registration, anatomical masking, temporal windowing, epoch segmentation, or normalization. In certain embodiments, coefficient generation is performed on a rolling basis, on a windowed basis, on a trial basis, or on another selected temporal schedule.

In certain embodiments, where sensor geometry is regular or partially regular, one or more discrete projection operations may be accelerated using a fast Fourier transform or another structured transform. Such acceleration is optional and does not alter the underlying use of the fixed Recognition-Physics-derived cortical eigen-basis.

In preferred embodiments, the output of the projection pipeline includes at least a coefficient vector w(t). In certain embodiments, the output further includes a ranked coefficient list, a projected-state record, a reconstruction quality measure, a residual magnitude, a normalization status, or another quality-control quantity suitable for downstream readout, control, validation, or storage.

In certain embodiments, the projection pipeline is configured to output coefficients at a rate sufficient to support real-time or near-real-time readout and, in write-capable embodiments, basis-consistent stimulation control.

In preferred embodiments, deterministic readout subsystem 118 receives coefficient vector w(t) and determines a qualia state according to a deterministic mapping defined over the disclosed cortical eigen-basis. In such embodiments, the mapping from coefficient vector to qualia state does not depend on subject-specific learned classifier weights, subject-specific training labels, or subject-specific statistical decoder training.

In some embodiments, the basis-defined output is not merely displayed for human interpretation, but is consumed by downstream machine logic that selects a stimulation pattern, updates a controller state, determines whether a target-state command is accepted, blocks or permits a write operation, stores a basis-defined qualia track, triggers a safety response, or routes the coefficient-space output to a downstream prosthetic, diagnostic, immersive-media, or monitoring process. Accordingly, the disclosed coefficient-space representation forms part of a machine-control workflow rather than merely constituting a passive report of neural activity.

In one preferred implementation, deterministic readout subsystem 118 computes a magnitude or absolute value associated with each coefficient w_k(t), ranks the coefficients, identifies a dominant mode index k*, and maps the dominant mode index to a qualia category according to a fixed qualia table.

In one preferred implementation, the dominant mode index is determined according to the following expression:

k*=argmax_k|w_k(t)|

where k* denotes the index of the coefficient having the greatest magnitude within the disclosed basis.

In preferred embodiments using the eight-mode implementation described herein, the fixed qualia table associates the dominant mode index with a corresponding qualia category. In one preferred mapping, mode 0 corresponds to visual qualia, mode 1 corresponds to auditory qualia, mode 2 corresponds to somatosensory qualia, mode 3 corresponds to nociceptive qualia, mode 4 corresponds to emotional qualia, mode 5 corresponds to proprioceptive qualia, mode 6 corresponds to gustatory qualia, and mode 7 corresponds to olfactory qualia.

In preferred embodiments, deterministic readout subsystem 118 outputs at least a dominant qualia class determined from the dominant mode index. In certain embodiments, deterministic readout subsystem 118 further outputs one or more coefficient values, a ranked coefficient list, a time stamp, a normalization value, a residual value, a sub-classification output, or another report object associated with the determined qualia state.

In certain embodiments, sub-classification is performed within a dominant qualia category using one or more secondary coefficients, tertiary coefficients, coefficient phases, coefficient signs, temporal patterns, or additional basis-defined features. Such sub-classification may be used to refine a category-level output without altering the primary deterministic basis mapping.

By way of non-limiting example, in a visual embodiment, magnitude associated with a dominant visual-mode coefficient may correspond to brightness or intensity, while phase information may correspond to hue or another visual attribute. In an auditory embodiment, magnitude associated with a dominant auditory-mode coefficient may correspond to loudness, while temporal or oscillatory structure may correspond to pitch or tonal quality. In an emotional embodiment, sign and magnitude associated with an emotional-mode coefficient may correspond to valence and arousal, respectively.

In preferred embodiments, deterministic readout subsystem 118 operates on the basis-defined coefficient vector rather than on a subject-specific learned latent space. Accordingly, the disclosed interface provides a non-statistical representational basis for qualia readout in which category identification is determined by basis-defined structure rather than by post hoc learned classifier fitting.

In certain embodiments, a qualia output may be stored as a basis-defined state record comprising the coefficient vector, a dominant-mode classification, optional sub-classification information, normalization data, and one or more time stamps. Such stored output may be used for review, replay, validation, comparison across sessions, or downstream application processing.

In preferred embodiments, deterministic readout subsystem 118 is configured such that the same coefficient vector yields the same qualia-state identification under the disclosed mapping. In this sense, the readout process is deterministic with respect to the basis-defined coefficient representation.

In preferred embodiments, the disclosed interface is training-free with respect to qualia classification. More particularly, no subject-specific training data are used to learn a mapping between neural measurements and qualia categories. Instead, the disclosed architecture derives its readout from a fixed Recognition-Physics-derived cortical eigen-basis, a basis-defined coefficient representation, and a deterministic qualia mapping defined over that basis.

In some embodiments, training-free operation refers specifically to the absence of subject-specific supervised learning of a qualia classifier or decoder and does not preclude subject-specific anatomical registration, signal-quality screening, coupling calibration, threshold configuration, safety-limit selection, or validation procedures needed to instantiate or operate a particular embodiment. In this manner, the disclosed architecture distinguishes qualia-model training from physical-system instantiation and operational qualification.

In preferred embodiments, the eigen-modes are determined by the projected Recognition-Physics operator, the anatomical embedding is determined by subject anatomy, and the qualia mapping is determined by the disclosed mode structure. The disclosed interface therefore does not require subject-specific supervised training of a qualia decoder.

In certain embodiments, subject-specific anatomical data are used to map universal eigen-modes onto a subject-specific cortical geometry, and subject-specific coupling calibration data are used for write-path synthesis. Such measurements do not constitute qualia-model training and do not change the fixed basis-defined mapping between coefficient-space structure and qualia categories.

In certain embodiments, a read operation may be performed in a read-only mode in which cortical neural activity is acquired, projected into the disclosed basis, and interpreted as a qualia state without any stimulation output. In such embodiments, the system may provide coefficient-vector output, dominant qualia output, optional sub-classification output, and one or more validation or quality-control outputs.

In preferred embodiments, readout performance may be evaluated using one or more validation metrics. By way of non-limiting example, read fidelity may be defined as the probability of correct qualia-category identification conditioned on the coefficient vector. In preferred implementations, a target read fidelity of at least 0.90 may be used as a design or validation objective.

In certain embodiments, deterministic readout subsystem 118 may operate continuously, periodically, over selected windows, over selected epochs, or in response to one or more triggers. In certain embodiments, readout results may be used directly for reporting, monitoring, control, storage, validation, or generation of target states for subsequent write operations.

In write-capable embodiments, stimulation subsystem 124 is configured to generate cortical stimulation corresponding to a target qualia state represented in the same cortical eigen-basis used for readout. In preferred embodiments, a target qualia state is represented by a target coefficient vector w_target.

In preferred embodiments, target coefficient vector w_target specifies one or more desired coefficient values associated with one or more projected cortical eigen-modes. In certain embodiments, target coefficient vector w_target is provided directly by an operator, generated from a stored target-state library, generated from a prior readout, generated from an application-level input, generated from an external device signal, or generated from another process that produces a basis-defined target state.

In one preferred implementation, stimulation subsystem 124 generates a stimulation signal according to the following expression:

I(x,t)=sum_k s_k(t) psi_k\hat{ }cortex(x)

where psi_k\hat{ }cortex(x) denotes a projected cortical eigen-mode and s_k(t) denotes a stimulation weight, gain-adjusted coefficient, or other basis-specific drive value associated with mode k.

In one preferred implementation, the stimulation weight is determined according to the following expression:

s_k(t)=G_k*w_target_k(t)

where G_k denotes a mode-specific coupling quantity and w_target_k(t) denotes a target coefficient value for mode k.

In certain embodiments, the mode-specific coupling quantities are represented collectively by a coupling matrix G. In preferred embodiments, coupling matrix G is determined by a subject-specific calibration process and is subsequently stored for use in later stimulation sessions.

In certain embodiments, stimulation subsystem 124 receives as input a target qualia command, a target coefficient vector, cortical mode-pattern information, and one or more calibration quantities. In response thereto, stimulation subsystem 124 outputs one or more stimulation signals, waveforms, patterns, drive values, channel-specific settings, or other stimulation-control quantities suitable for application to cortical tissue or to a stimulation arrangement coupled to cortical tissue.

In certain embodiments, stimulation is delivered electrically. In certain embodiments, stimulation is delivered optically. In certain embodiments, stimulation is delivered magnetically. In certain embodiments, stimulation is delivered ultrasonically. In certain embodiments, stimulation is delivered using another cortical stimulation arrangement capable of approximating a desired basis-defined cortical pattern.

In preferred embodiments, write operations are performed in the same basis used for read operations. Accordingly, a qualia state determined in the disclosed coefficient-space representation may be reproduced, approximated, shifted, enhanced, reduced, or otherwise modulated using basis-consistent stimulation in the corresponding projected cortical eigen-basis.

In certain embodiments, a target qualia state may correspond to a dominant single-mode target. In certain embodiments, a target qualia state may correspond to a mixed-state target having multiple nonzero coefficients. In certain embodiments, a target qualia state may vary over time. In certain embodiments, stimulation subsystem 124 may generate amplitude-coded stimulation, phase-coded stimulation, time-varying stimulation, or combinations thereof, provided that the stimulation remains basis-consistent with the disclosed cortical eigen-basis.

In one preferred implementation, calibration subsystem 122 performs a one-time subject-specific coupling calibration. In such embodiments, a known stimulation pattern is applied along a selected projected cortical eigen-mode, a resulting coefficient response is measured through the read channel, and a coupling quantity is determined from the relationship between applied stimulation and measured coefficient response.

In one preferred calibration procedure, the following operations are performed: a known stimulation pattern corresponding to a selected mode psi_k is applied; a resulting coefficient response w_k is measured; a coupling value G_k is computed from the applied stimulation and measured response; and the resulting coupling value is stored for subsequent use. In one preferred expression:

G_k=I_applied_k/w_measured_k

where I_applied_k denotes an applied mode-specific stimulation quantity and w_measured_k denotes a measured response coefficient.

In certain embodiments, coupling calibration depends on one or more subject-specific physical properties, including electrode impedance, cortical thickness, tissue conductivity, optical response, stimulation geometry, or other anatomically or physically relevant factors. In preferred embodiments, such calibration remains distinct from qualia-model training because it characterizes physical coupling rather than learning a subject-specific qualia-classification rule.

In certain embodiments, calibration subsystem 122 stores one or more calibration products including coupling matrix G, mode-specific gain values, embedding data, calibration quality values, drift indicators, recalibration indicators, or combinations thereof. In certain embodiments, recalibration is performed periodically, conditionally, partially, or upon detection of one or more quality-control events.

In certain embodiments, the disclosed system operates in a write-only mode in which a target coefficient vector is received and stimulation is synthesized without an immediately preceding readout. In preferred embodiments, however, write operations may also be performed in conjunction with read operations such that the read channel and write channel operate over the same disclosed basis.

In preferred embodiments, controller 126 is configured to operate the disclosed interface in a closed-loop mode. In such embodiments, a measured coefficient vector is compared to a target coefficient vector, one or more update quantities are determined based on the difference between the two, and stimulation is adjusted accordingly.

In one preferred implementation, an error vector is computed according to the following expression:

e(t)=w_target-w(t)

where w_target denotes a target coefficient vector and w(t) denotes a measured coefficient vector at time t.

In preferred embodiments, the measured coefficient vector is normalized prior to control update. In one preferred implementation, normalization is represented as follows:

w_normalized=w/∥w∥

where∥w∥denotes a norm of the measured coefficient vector.

In preferred embodiments, the normalization convention is associated with a unit-sphere or unitarity constraint. In one preferred implementation, the normalized state satisfies:

sum_k|w_k(t)|\hat{ }2=1

Such normalization may provide a stable state convention for readout, control, write synthesis, and safety monitoring.

In one preferred implementation, a stimulation update is determined according to the following expression:

s(t)=G*(w_target +K_p e(t))

where K_p denotes a proportional control gain and G denotes a coupling matrix or equivalent calibration quantity.

In preferred embodiments, proportional gain K_p is selected to provide stable convergence toward a target coefficient vector while limiting oscillation, overshoot, or instability. In one preferred implementation, K_p has a default value of approximately 0.5, although other gain values may be used.

In certain embodiments, controller 126 performs a repeated sequence comprising: acquire a current neural state; compute a current coefficient vector; compare the current coefficient vector to a target coefficient vector; normalize one or more coefficient quantities; compute one or more update quantities; generate an updated stimulation command; and deliver stimulation. In preferred embodiments, this repeated sequence is performed at a rate suitable for real-time or near-real-time closed-loop operation.

In one preferred implementation, the disclosed closed-loop system operates at approximately 200 Hz with a cycle time of approximately 5 milliseconds. In other embodiments, higher or lower update rates may be used.

In certain embodiments, safety monitor 128 evaluates one or more safety conditions during operation of the disclosed interface. Such safety conditions may include stimulation current limits, optical power limits, magnetic field limits, coefficient normalization deviation, excessive residual magnitude, coupling faults, hardware faults, timing faults, drift conditions, or other conditions relevant to safe operation.

In preferred embodiments, one or more maximum stimulation limits are enforced. By way of non-limiting example, a maximum electrical current per stimulation channel, a maximum optical power density, or a maximum magnetic-field quantity may be specified for a given implementation. In certain embodiments, a protective action is triggered if a measured coefficient vector departs from a normalized state convention by more than a selected threshold.

In certain embodiments, the system enters a reduced-output mode, a paused mode, a shutdown mode, or a sham mode upon occurrence of a safety event. In certain embodiments, safety monitor 128 records one or more safety events in an event log for later review, validation, or regulatory documentation.

In certain embodiments, write performance is evaluated using one or more validation metrics. By way of non-limiting example, write fidelity may be defined according to an overlap quantity between a measured coefficient vector and a target coefficient vector. In preferred implementations, a target write fidelity of at least 0.80 may be used as a design or validation objective.

In certain embodiments, stimulation subsystem 124, calibration subsystem 122, controller 126, and safety monitor 128 cooperate to provide a basis-consistent read/write qualia interface in which target states are specified, generated, monitored, and adjusted in the same disclosed cortical eigen-basis used for deterministic qualia readout.

In certain embodiments, system 100 is implemented using one or more sensing devices, one or more stimulation devices, and one or more processors configured to execute preprocessing, basis projection, readout, calibration, control, safety, storage, and user-interface operations. In certain embodiments, compute hardware 134 comprises one or more processors, digital signal processors, field-programmable gate arrays, graphics processors, application-specific integrated circuits, memory devices, storage devices, or combinations thereof.

In certain embodiments, basis datastore 112 stores one or more basis definitions, mode patterns, eigenvalue ordering data, projector data, anatomical embedding data, calibration data, or combinations thereof. In certain embodiments, output datastore 130 stores coefficient vectors, qualia classifications, target coefficient vectors, stimulation waveforms, validation data, safety logs, event logs, qualia tracks, and other output products generated by the disclosed interface.

In certain embodiments, user interface 132 receives target-state inputs, stimulation-session inputs, validation inputs, control inputs, or storage and replay inputs. In certain embodiments, user interface 132 displays one or more current coefficient values, dominant-mode identifications, qualia-state outputs, stimulation settings, calibration information, warning indicators, validation metrics, or stored records.

In certain embodiments, the disclosed interface is implemented as an invasive system. By way of non-limiting example, neural acquisition subsystem 104 may comprise a subdural electrocorticography array, and stimulation subsystem 124 may comprise one or more electrical stimulation channels configured to apply cortical stimulation according to basis-defined stimulation patterns.

In certain embodiments, the disclosed interface is implemented as a non-invasive system. By way of non-limiting example, neural acquisition subsystem 104 may comprise electroencephalography, magnetoencephalography, functional near-infrared spectroscopy, or combinations thereof, and stimulation subsystem 124 may comprise transcranial magnetic stimulation, ultrasound stimulation, optical stimulation, or another non-invasive or minimally invasive stimulation arrangement capable of approximating a basis-defined cortical pattern.

In certain embodiments, the disclosed interface is implemented using an all-optical arrangement in which one or more optical sensing techniques are used for read operations and one or more optical stimulation techniques are used for write operations. In certain embodiments, the disclosed interface is implemented using an intracortical array arrangement. In certain embodiments, the disclosed interface is implemented in an animal-model configuration. In certain embodiments, the disclosed interface is implemented in a reduced-channel or wearable configuration.

The foregoing hardware and implementation examples are representative only and do not redefine the core architecture, which remains a fixed-basis, training-free, deterministic qualia interface using a Recognition-Physics-derived cortical eigen-basis and, in write-capable embodiments, same-basis stimulation.

The following embodiments are downstream applications of the disclosed interface and do not redefine the core architecture.

In one downstream embodiment, the disclosed interface is used for pain modulation. In such an embodiment, a nociceptive-mode coefficient may be measured, a target state may be defined in which the nociceptive-mode coefficient is reduced relative to a baseline state, and stimulation may be applied in the disclosed basis to reduce the nociceptive component while preserving one or more additional coefficient components.

In one downstream embodiment, the disclosed interface is used for neuro-prosthetic feedback. In such an embodiment, an external device signal, including but not limited to a tactile, force, temperature, pressure, or position signal associated with a prosthetic device, is converted into a target coefficient vector corresponding to one or more somatosensory or proprioceptive modes, and the target coefficient vector is written to the subject using basis-consistent stimulation.

In one downstream embodiment, the disclosed interface is used for immersive-media or authoring applications. In such an embodiment, a coefficient vector associated with a desired sensory or affective state is generated, stored, synchronized, replayed, or written as part of a basis-defined qualia record or qualia track.

In one downstream embodiment, the disclosed interface is used for anesthesia monitoring, neurological monitoring, or diagnosis. In such an embodiment, one or more coefficient vectors, residual values, normalization measures, or mode-specific variations may be monitored over time and compared to one or more reference states, thresholds, or expected conditions.

In one downstream embodiment, the disclosed interface is used for emotional-state induction, immersive virtual-reality enhancement, or another experiential-modulation application in which one or more target coefficients are selected to induce, reinforce, suppress, or stabilize a selected basis-defined state.

In certain embodiments, one or more output products generated by the disclosed interface are stored, transmitted, replayed, compared across sessions, compared across subjects, or used to generate a validation or control record. In certain embodiments, such output products include one or more coefficient vectors, target coefficient vectors, stimulation patterns, calibration products, validation reports, safety reports, or qualia tracks.

In certain embodiments, the disclosed interface is validated using a visual-qualia decoding protocol. In one example of such a protocol, one or more visual stimuli are presented to a subject, cortical neural data are acquired, the acquired data are projected into the disclosed basis, a dominant or otherwise relevant coefficient structure is determined, and the resulting basis-defined output is compared with a corresponding expected or reported state.

In certain embodiments, the disclosed interface is validated using an auditory-write protocol. In one example of such a protocol, a target auditory-state coefficient vector is defined, a basis-consistent stimulation pattern is generated, a resulting neural response or reported state is measured, and one or more overlap or detection metrics are calculated.

In certain embodiments, the disclosed interface is validated using a sham-controlled crossover protocol. In one example of such a protocol, active stimulation and sham stimulation are selectively applied in different sessions, intervals, or blinded conditions, and resulting coefficient, report, detection, or behavioral outputs are compared to evaluate whether the observed effect exceeds a sham baseline.

In certain embodiments, the disclosed interface is validated using a closed-loop stability protocol. In one example of such a protocol, a target coefficient vector is defined, the disclosed interface is operated in a closed-loop mode, stimulation is updated based on measured error relative to the target state, and stability, drift, residual magnitude, normalization deviation, or fidelity metrics are evaluated over time.

In certain embodiments, the disclosed interface is validated using a cross-subject universality protocol. In one example of such a protocol, the same basis-defined mapping is applied to multiple subjects, while allowing subject-specific anatomical embedding and coupling calibration, and resulting qualia-classification or state-control outputs are compared to assess cross-subject portability of the disclosed basis-defined readout architecture.

In preferred validation implementations, one or more acceptance criteria may be used, including one or more target read-fidelity values, target write-fidelity values, false-positive thresholds, drift thresholds, stability thresholds, residual thresholds, safety thresholds, or cross-subject performance thresholds. Such validation thresholds may be used as design objectives, test objectives, benchmarking objectives, or acceptance objectives for a particular embodiment or deployment.

In certain embodiments, one or more validation processes are performed in a human subject, an animal subject, a simulated environment, a benchtop environment, a hybrid environment, or another evaluation environment suitable for testing one or more aspects of the disclosed interface.

In certain embodiments, one or more embodiments described herein are illustrative, one or more embodiments are representative implementations, one or more embodiments are downstream applications, and one or more embodiments are prophetic or future-facing. The disclosed specification is intended to preserve the core invention, preferred implementations, optional implementations, and continuation-worthy future variations while maintaining the identity of the invention as a fixed-basis, training-free qualia read/write interface.

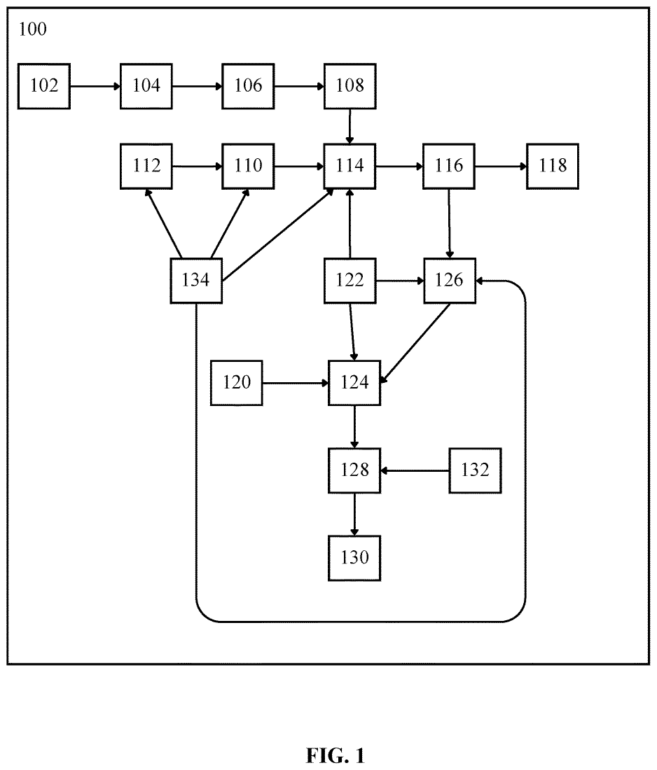

FIG. 1 depicts an overall block diagram of an Eigen-Cluster Neuro-Interface system 100. As shown, system 100 may include subject cortex 102, neural sensor array 104, acquisition frontend 106, preprocessing module 108, covariance engine 110, Recognition-Physics basis datastore 112, projection engine 114, coefficient vector output 116, deterministic readout module 118, target qualia input 120, calibration store 122, stimulation synthesis module 124, controller 126, safety monitor 128, output datastore 130, user interface 132, and compute hardware 134.

In FIG. 1, neural sensor array 104 obtains neural data from subject cortex 102 and provides such data to acquisition frontend 106. Acquisition frontend 106 provides acquired data to preprocessing module 108, which in turn provides preprocessed data to covariance engine 110 and projection engine 114. Recognition-Physics basis datastore 112 provides basis information to projection engine 114. Projection engine 114 generates coefficient vector output 116, which is provided to deterministic readout module 118 and, in write-capable embodiments, to controller 126. Target qualia input 120 provides a target-state input to controller 126 and/or stimulation synthesis module 124. Calibration store 122 provides anatomical embedding data, coupling calibration data, or both to projection engine 114, stimulation synthesis module 124, and controller 126. Stimulation synthesis module 124 generates one or more stimulation outputs, which are monitored by safety monitor 128. Output datastore 130 stores coefficient vectors, classifications, stimulation records, calibration records, safety logs, qualia tracks, and other output products.

FIG. 2 depicts a read workflow 200. Read workflow 200 may include neural data input 202, preprocessing stage 204, covariance stage 206, basis projection stage 208, coefficient generation stage 210, dominant-mode selector 212, fixed lookup table 214, sub-classification stage 216, and qualia output object 218. In operation, neural data input 202 is processed by preprocessing stage 204, basis-related quantities are generated or updated at covariance stage 206, basis projection stage 208 projects the data into the disclosed cortical eigen-basis, coefficient generation stage 210 produces one or more coefficients, dominant-mode selector 212 identifies a dominant mode, fixed lookup table 214 maps the dominant mode to a qualia category, and sub-classification stage 216 optionally refines the category-level output before qualia output object 218 is generated.

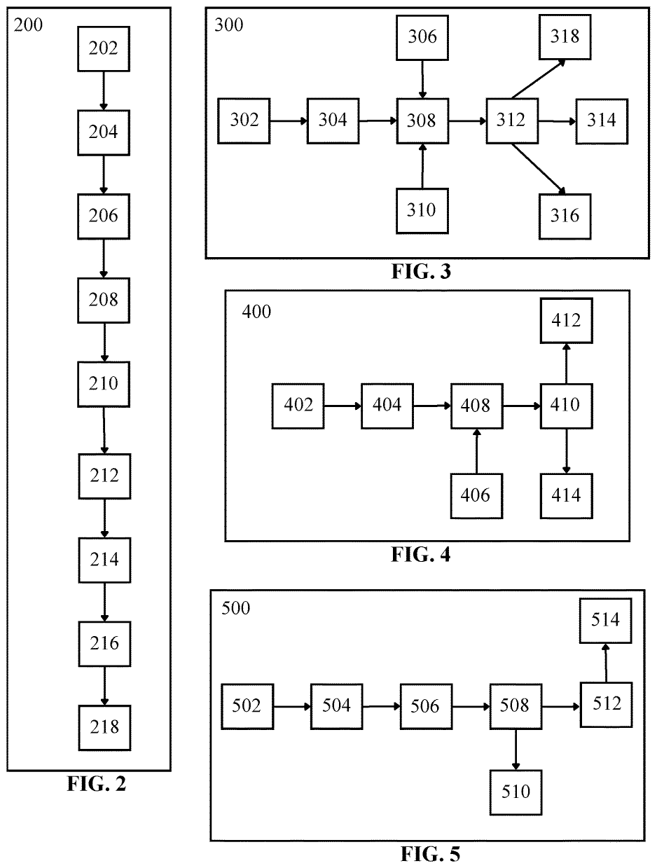

FIG. 3 depicts an extraction architecture 300 for eigen-cluster extraction and joint diagonalization. Extraction architecture 300 may include sensor-space matrix 302, covariance matrix C 304, Recognition-Physics basis 306, joint diagonalization engine 308, orthogonality constraint input 310, transform matrix W 312, coefficient vector w(t) 314, residual path 316, and quality metric output 318. Sensor-space matrix 302 is used to generate covariance matrix C 304. Joint diagonalization engine 308 receives covariance matrix C 304, Recognition-Physics basis 306, and orthogonality constraint input 310 to determine transform matrix W 312. Transform matrix W 312 is used to generate coefficient vector w(t) 314. Residual path 316 may carry a reconstruction residual, out-of-basis activity, or orthogonal complement quantity. Quality metric output 318 may include a residual magnitude, normalization value, projection quality score, or another metric suitable for validation, control, or safety use.

FIG. 4 depicts embedding architecture 400. Embedding architecture 400 may include abstract mode space 402, universal modes 404, anatomy input 406, embedding transform iota 408, cortical manifold 410, projected cortical modes 412, and stored embedding data 414. In operation, abstract mode space 402 and universal modes 404 are mapped to cortical manifold 410 using embedding transform iota 408 determined from anatomy input 406. Stored embedding data 414 may retain one or more resulting mappings, transforms, surfaces, or other subject-specific geometric quantities used to instantiate projected cortical modes 412 on a subject cortex.

FIG. 5 depicts readout logic 500. Readout logic 500 may include coefficient input 502, magnitude ranking 504, dominant-mode output 506, fixed qualia table 508, optional refinement block 510, qualia report 512, and optional coefficient archive 514. Coefficient input 502 supplies a coefficient vector to magnitude ranking 504. Magnitude ranking 504 provides a ranked result to dominant-mode output 506, which identifies a dominant mode. Fixed qualia table 508 maps the dominant mode to a qualia category. Optional refinement block 510 may refine that result using additional coefficient information. Qualia report 512 may include a category, sub-category, coefficient record, time stamp, or other output. Optional coefficient archive 514 may store one or more basis-defined records associated with the read result.

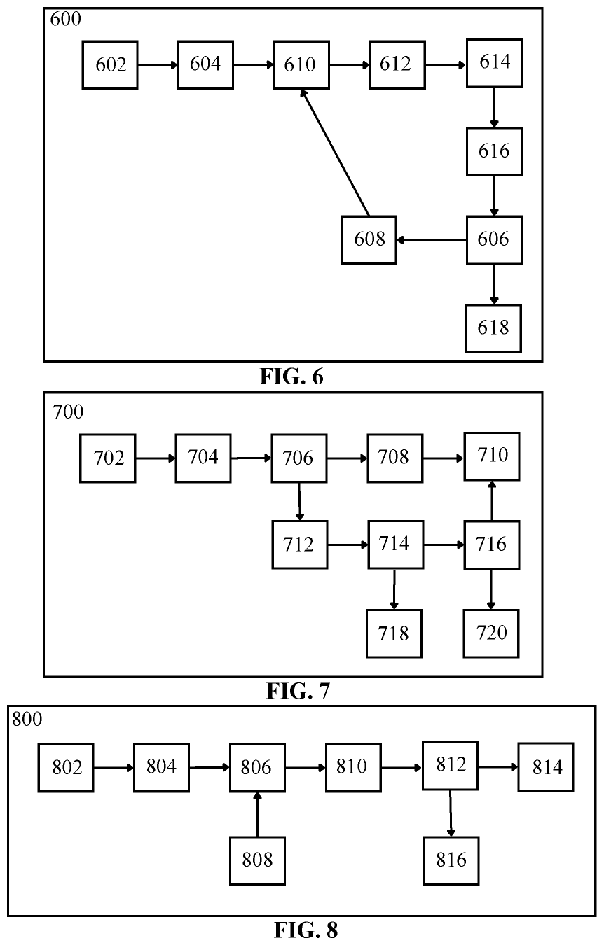

FIG. 6 depicts write architecture 600. Write architecture 600 may include target qualia input 602, target coefficient vector 604, calibration engine 606, coupling matrix G 608, basis-pattern synthesis 610, stimulation driver 612, cortical stimulation output 614, measured response feedback 616, and stored calibration record 618. Target qualia input 602 provides a desired state to target coefficient vector 604. Calibration engine 606 generates or updates coupling matrix G 608 using measured response feedback 616. Basis-pattern synthesis 610 uses target coefficient vector 604 and coupling matrix G 608 to produce one or more drive values provided to stimulation driver 612. Stimulation driver 612 provides cortical stimulation output 614. Stored calibration record 618 may retain one or more calibrated quantities for later sessions.

FIG. 7 depicts closed-loop control 700. Closed-loop control 700 may include current state input 702, error computation 704, normalization block 706, gain update block 708, stimulation command 710, unitarity monitor 712, threshold comparator 714, shutdown gate 716, sham selector 718, and safety log 720. Current state input 702 provides a measured coefficient vector to error computation 704, which compares the measured vector to a target vector. Normalization block 706 applies a normalized state convention. Gain update block 708 determines an updated stimulation command 710. Unitarity monitor 712 and threshold comparator 714 monitor one or more quality or safety conditions. Shutdown gate 716 may limit or stop stimulation under selected conditions. Sham selector 718 may provide blinded or control-condition operation. Safety log 720 may store warnings, limits, events, and actions.

FIG. 8 depicts mathematical framework 800. Mathematical framework 800 may include Recognition-Physics operator H_RP 802, projected cortical operator H_cortex 804, eigen-equation block 806, eigenvalue ordering 808, rank-8 basis subspace 810, projector Pi_8 812, projected component u_8 814, and orthogonal residual u_perp 816. Recognition-Physics operator H_RP 802 is mapped to projected cortical operator H_cortex 804. Eigen-equation block 806 represents the mode relationship used to define projected cortical eigen-modes. Eigenvalue ordering 808 represents the preferred mode-ordering structure. Rank-8 basis subspace 810 represents the preferred disclosed eight-mode implementation. Projector Pi_8 812 represents projection into the disclosed subspace. Projected component u_8 814 and orthogonal residual u_perp 816 represent basis-contained and out-of-basis components, respectively.

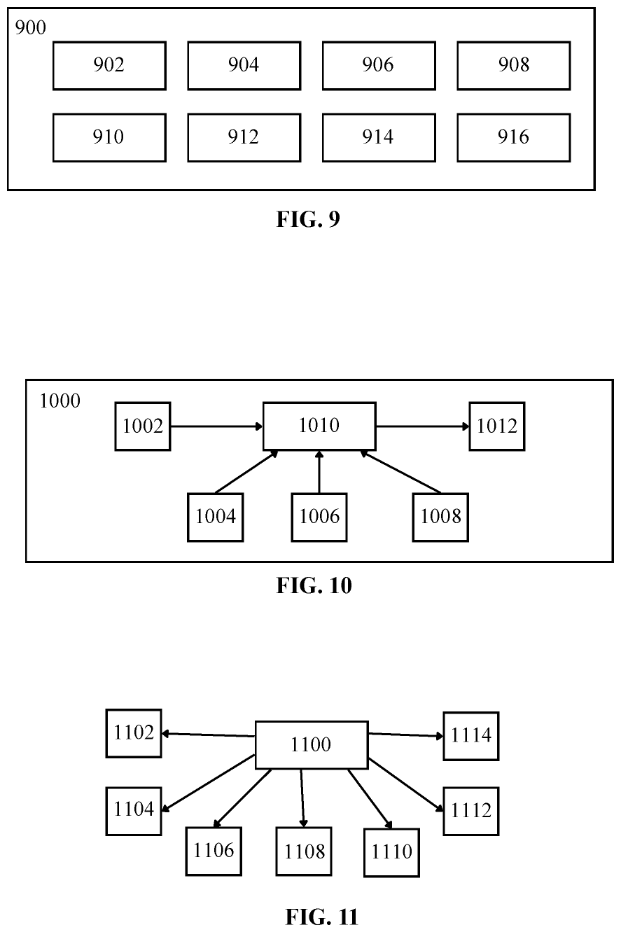

FIG. 9 depicts embodiment panel 900 showing representative hardware embodiments. Embodiment panel 900 may include ECoG embodiment 902, MEG embodiment 904, EEG embodiment 906, fNIRS embodiment 908, TMS embodiment 910, ultrasound embodiment 912, optical embodiment 914, and intracortical embodiment 916. The embodiments shown in FIG. 9 are representative sensing and stimulation arrangements that may be used with the disclosed fixed-basis qualia interface. The inclusion of such embodiments does not alter the core architecture described herein.

FIG. 10 depicts downstream embodiments 1000. Downstream embodiments 1000 may include pain modulation path 1002, prosthetic feedback path 1004, immersive media path 1006, anesthesia/diagnostic path 1008, target-state conversion block 1010, and subordinate output block 1012. Pain modulation path 1002 represents a downstream embodiment in which one or more nociceptive-related coefficients are reduced or modulated. Prosthetic feedback path 1004 represents a downstream embodiment in which external device data are converted into one or more target coefficients. Immersive media path 1006 represents a downstream embodiment in which one or more basis-defined states are stored, replayed, synchronized, or authored. Anesthesia/diagnostic path 1008 represents a downstream embodiment in which basis-defined quantities are monitored over time. Target-state conversion block 1010 and subordinate output block 1012 represent transformation of downstream application inputs into basis-defined targets and related outputs.

FIG. 11 depicts support architecture 1100. Support architecture 1100 may include core invention box 1102, preferred embodiments box 1104, optional embodiments box 1106, prophetic material box 1108, formal verification box 1110, validation protocol box 1112, and continuation targets box 1114. FIG. 11 illustrates separation between the core disclosed invention, narrower preferred implementations, optional engineering embodiments, prophetic material, formal model-level verification, validation protocols, and future continuation opportunities. Such separation supports consistency, written-description discipline, and claim-architecture discipline across the application.

The figures described above are illustrative and are not intended to limit the disclosed subject matter to any particular arrangement unless expressly recited in the claims. Components shown in one figure may be used with components shown in another figure, omitted, rearranged, combined, subdivided, repeated, or otherwise modified in accordance with the teachings of the present disclosure.

In certain embodiments, the disclosed interface is supported by formal verification and machine-checked properties. In preferred implementations, such formally verified properties include orthonormality of basis functions, normalization or unitarity enforcement, deterministic decomposition of a neural field into coefficient-space representation, deterministic readout from coefficient representation to qualia-state output, linear write synthesis, preferred closed-loop rate properties, preferred cycle-time properties, parameter-free basis structure in the disclosed implementation, and sensor-agnostic operation across multiple supported sensing modalities.

In certain embodiments, such formal verification is generated using a machine-checked proof environment and is used to support the internal mathematical consistency, control logic, and structural properties of the disclosed interface. Such formal verification is distinct from empirical human-subject validation and should not be construed as replacing clinical, benchtop, animal-model, or other experimental validation procedures.

In certain embodiments, the disclosed interface generates one or more stored output products or stored artifacts. By way of non-limiting example, such stored output products may include a coefficient-vector record representing a measured qualia state, a target coefficient-vector record representing a target qualia state, a stored cortical embedding file, a stored coupling matrix or calibration record, a generated stimulation waveform or stimulation-pattern record, a validation report, a safety report, or a qualia track.

In certain embodiments, a qualia track comprises a stored basis-defined representation of one or more coefficient vectors, target vectors, stimulation commands, time stamps, metadata, or combinations thereof, such that a previously measured, authored, generated, or validated state may later be reviewed, replayed, analyzed, compared, synchronized, or used to generate a later stimulation process.

In certain embodiments, one or more stored artifacts generated by the disclosed interface are retained in output datastore 130, calibration store 122, or another storage location coupled to system 100. In certain embodiments, such stored artifacts are used to support repeated-session operation, validation, calibration reuse, cross-session comparison, downstream application processing, control refinement, or archival record keeping.

In certain embodiments, one or more future-facing or continuation-worthy embodiments are contemplated. Such embodiments may include multi-subject coupling, direct qualia transfer, expanded mode systems beyond the disclosed eight-mode implementation, reduced-channel consumer-device variants, richer qualia-track generation and replay systems, and specialized bio-write implementations including optically mediated or biologically specialized stimulation arrangements. Such embodiments are disclosed to preserve future flexibility and continuation support and do not redefine the core architecture disclosed herein.

In certain embodiments, multi-subject coupling may include reading a basis-defined coefficient state from a first subject, transmitting a representation of that coefficient state or a derived target state, and generating stimulation in a second subject according to the same or a corresponding basis-defined representation. In certain embodiments, such embodiments are prophetic or future-facing and are not required for practicing the core disclosed interface.

In certain embodiments, expanded mode systems may include additional basis elements, additional subspaces, hierarchical mode sets, non-human cortical mappings, or other extensions of the disclosed basis-defined approach. In certain embodiments, such expanded systems remain within the disclosed architectural concept to the extent that cortical neural activity is represented in a fixed Recognition-Physics-derived basis and read or written according to basis-defined coefficient structure.

The present specification preserves multiple support levels. The core invention is the fixed-basis, training-free, deterministic qualia interface described herein. Preferred embodiments include, for example, the eight-mode implementation, MRI-derived anatomical embedding, one-time coupling calibration, and closed-loop normalization. Optional and alternative embodiments include modality-specific sensing and stimulation implementations. Validation material includes the protocols and target metrics described herein. Prophetic material includes future-facing embodiments such as multi-subject transfer and certain continuation targets. Downstream embodiments include application-layer uses such as pain modulation, prosthetic feedback, immersive media, monitoring, and diagnosis.

Accordingly, the present disclosure is intended to preserve a clear and defensible statement of the core invention while also preserving narrower fallback positions, optional implementations, validation pathways, downstream application support, and future continuation support, without allowing optional or prophetic material to swallow the identity of the invention.

In preferred embodiments, the disclosed cortical eigen-basis is not merely a selected statistical representation, latent-space decomposition, principal-component basis, independent-component basis, or other empirically chosen signal-analysis coordinate system. Rather, the disclosed cortical eigen-basis is defined by the Recognition-Physics framework and is used as an integral part of the technical operation of the interface for coefficient generation, deterministic qualia readout, target-state definition, stimulation synthesis, normalization, and closed-loop control.

In the disclosed architecture, the mode structure is integral to the interface's technical function and is not presented merely as one interchangeable decomposition among many. More particularly, the disclosed system operates by acquiring cortical neural data, projecting the data into the disclosed Recognition-Physics-derived cortical eigen-basis, determining basis-defined coefficients, mapping those coefficients to qualia-state output according to a deterministic basis-defined mapping, and, in write-capable embodiments, generating stimulation in the same basis. Accordingly, the disclosed basis is part of the machine architecture itself and not merely a post hoc analytical labeling scheme.

In certain embodiments, the formal verification described herein is implemented in a machine-checked proof environment, including a Lean proof environment, using a proof artifact corresponding to the disclosed Recognition-Neuro-Interface architecture. Such proof artifacts may verify, by way of non-limiting example, orthonormality, deterministic decomposition, deterministic readout, linear write synthesis, normalization properties, rate properties, and basis-structure properties of the disclosed interface. Such formal verification supports the internal technical consistency of the machine implementation while remaining distinct from empirical validation of clinical or human-subject performance.

By way of a first illustrative and non-limiting worked example, a visual-state read operation may be performed by acquiring cortical neural data from a subject during presentation of a visual stimulus, projecting the acquired data into the disclosed cortical eigen-basis, determining a coefficient vector, identifying a dominant mode index, and outputting a visual qualia classification from the fixed qualia table. In certain embodiments, one or more secondary coefficients, magnitudes, or phases may be used to refine the visual-state output.

By way of a second illustrative and non-limiting worked example, an auditory-state write operation may be performed by defining a target coefficient vector corresponding to an auditory qualia state, determining one or more mode-specific stimulation weights using a stored coupling calibration, synthesizing a stimulation pattern in the disclosed cortical eigen-basis, and applying the stimulation pattern to cortical tissue. In certain embodiments, a resulting measured coefficient vector may be compared to the target coefficient vector to evaluate overlap, convergence, or write fidelity.

By way of a third illustrative and non-limiting worked example, a nociceptive-state modulation operation may be performed by determining a measured coefficient vector for a subject, identifying a nociceptive-related coefficient, defining a target coefficient vector in which the nociceptive-related coefficient is reduced relative to the measured state, and generating basis-consistent cortical stimulation to drive the measured state toward the target state while preserving one or more additional coefficient components.

What is claimed is:

1. A computer-implemented method of interfacing with subjective qualia, the method comprising:
obtaining cortical neural data representing a neural field from cortical tissue of a subject via one or more neural sensing modalities;
decomposing the cortical neural data into a plurality of coefficients in a fixed Recognition-Physics-derived cortical eigen-basis using projected cortical eigen-functions;
identifying a qualia state from the plurality of coefficients according to a deterministic mapping defined over the cortical eigen-basis, without subject-specific training data for qualia classification;
and generating a stimulation signal for writing a target qualia state in the same cortical eigen-basis.

2. The method of claim 1, wherein identifying the qualia state comprises identifying a dominant mode index from the plurality of coefficients and mapping the dominant mode index to a qualia category according to a fixed qualia table.

3. The method of claim 1, wherein the fixed Recognition-Physics-derived cortical eigen-basis comprises eight modes associated with an 8-tick Recognition structure.

4. The method of claim 3, wherein the eight modes correspond respectively to visual, auditory, somatosensory, nociceptive, emotional, proprioceptive, gustatory, and olfactory qualia categories.

5. The method of claim 1, wherein the cortical neural data are represented according to:
u(x,t)=sum_k w_k(t) psi_k(x).

6. The method of claim 1, wherein the plurality of coefficients is determined according to:
w_k(t)=integral over M_cortex of u(x,t) psi_k(x) dA(x).

7. The method of claim 1, further comprising mapping one or more universal eigen-modes onto a subject-specific cortical geometry using anatomical embedding data.

8. The method of claim 7, wherein the anatomical embedding data comprise magnetic resonance imaging data.

9. The method of claim 7, wherein subject-specific variation is limited to anatomical embedding and stimulation-coupling calibration.

10. The method of claim 1, wherein generating the stimulation signal comprises synthesizing a stimulation pattern according to:
I(x, t)=sum_k s_k(t) psi_k\hat{ }cortex(x).

11. The method of claim 10, wherein one or more stimulation weights are determined according to:
s_k(t)=G_k*w_target_k(t).

12. The method of claim 1, further comprising determining one or more subject-specific stimulation-coupling values by:
applying a known stimulation pattern corresponding to a selected basis mode;
measuring a resulting coefficient response; and
computing the one or more subject-specific stimulation-coupling values from the known stimulation pattern and the resulting coefficient response.

13. The method of claim 1, wherein the plurality of coefficients is normalized according to a unit-sphere or unitarity convention.

14. The method of claim 13, wherein the plurality of coefficients is normalized such that sum_k|w_k(t)|\hat{ }2=1.

15. The method of claim 1, further comprising operating in a closed-loop mode by:
determining a measured coefficient vector;
comparing the measured coefficient vector to a target coefficient vector;
determining an error quantity from the comparison; and
updating one or more stimulation commands based on the error quantity.

16. The method of claim 15, further comprising monitoring a normalization deviation, a residual quantity, or a stimulation threshold condition and limiting, pausing, or terminating stimulation upon occurrence of a safety event.

17. A brain-computer interface system comprising:
a neural acquisition subsystem configured to acquire cortical neural data representing a neural field from cortical tissue of a subject via one or more neural sensing modalities;
a basis datastore storing a fixed Recognition-Physics-derived cortical eigen-basis;
an eigen-decomposition subsystem configured to decompose the cortical neural data into a plurality of coefficients in the fixed Recognition-Physics-derived cortical eigen-basis;
a deterministic qualia readout subsystem configured to identify a qualia state from the plurality of coefficients according to a deterministic mapping defined over the cortical eigen-basis without subject-specific training data for qualia classification; and
a stimulation subsystem configured to generate a stimulation signal for writing a target qualia state in the same cortical eigen-basis.

18. The system of claim 17, wherein the fixed Recognition-Physics-derived cortical eigen-basis comprises eight modes associated with an 8-tick Recognition structure, and wherein the deterministic qualia readout subsystem is configured to identify a dominant mode index and map the dominant mode index to a qualia category according to a fixed qualia table.

19. The system of claim 17, further comprising a calibration subsystem configured to determine and store anatomical embedding data and stimulation-coupling calibration data, and a controller configured to operate the system in a closed-loop mode.

20. A non-transitory computer-readable medium storing instructions that, when executed by one or more processors, cause the one or more processors to:
obtain cortical neural data representing a neural field from cortical tissue of a subject via one or more neural sensing modalities;
decompose the cortical neural data into a plurality of coefficients in a fixed Recognition-Physics-derived cortical eigen-basis using projected cortical eigen-functions;
identify a qualia state from the plurality of coefficients according to a deterministic mapping defined over the cortical eigen-basis, without subject-specific training data for qualia classification; and
generate a stimulation signal for writing a target qualia state in the same cortical eigen-basis.
